(19) United States
(12) Patent Application Publication
(10) Pub. No.: US 2026/0144556 A1
(43) Pub. Date: May 28, 2026

(54) MECHANICAL ASPIRATION SYRINGE AND ASSOCIATED FLUID FLOW CONTROL DEVICES

(71) Applicant: INNOVA VASCULAR, INC., Irvine, CA (US)

(72) Inventors: Cheng Ian, Irvine, CA (US); James Dinh, Irvine, CA (US); Greg Bak-Boychuk, Irvine, CA (US); Paul Mullen, Irvine, CA (US); Sanjay Shrivastava, Irvine, CA (US)

(21) Appl. No.: 19/396,914

(22) Filed: Nov. 21, 2025

Related U.S. Application Data
(60) Provisional application No. 63/724,130, filed on Nov. 22, 2024.

(51) Int. Cl. A61B 17/22; A61B 17/00; A61M 1/00; A61M 39/22; A61M 39/24; F16K 15/14

(52) U.S. Cl. CPC A61B 17/22; A61M 1/815; A61M 39/24; F16K 15/147; A61B 2017/00407; A61B 2017/00561; A61B 2017/22079; A61M 2039/226; A61M 2039/229; A61M 2039/2426; A61M 2039/244; A61M 2039/2486; A61M 2039/2493; A61M 2202/0413

(57) ABSTRACT

A vacuum generating device (i.e., mechanical aspiration syringe) and associated fluid flow control devices for thrombectomy are provided. The mechanical aspiration syringe comprises a plunger operatively connected to a tension-generating member and ratcheting lever(s), and can be configured in a “primed” or “charged” state, whereupon vacuum activation via actuation of a ratchet locking member will release the tensioned plunger, thereby initiating aspiration of clot material into a chamber. Fluid control devices and related devices for use in thrombectomy are also provided, including fluid decoupling dampers, slit valve assemblies, gate valve assemblies, and plunger valve assemblies.

### CROSS-REFERENCE TO RELATED APPLICATIONS

This application is a non-provisional and claims benefit of U.S. Provisional Application No. 63/724,130 filed Nov. 22, 2024 the specification of which is incorporated herein in its entirety by reference.

### FIELD OF THE INVENTION

The present invention relates to the field of intravascular clot removal and thrombectomy, especially aspiration thrombectomy.

### BACKGROUND OF THE INVENTION

Thrombectomy procedures are regularly performed as a medical means of removing unwanted clot burden from a patient's vasculature. Catheter based thrombectomy procedures can basically be described as advancing a catheter through the patient's vasculature to a location where the clot material is to be extracted. Once at the desired location within the patient's vessel, the catheter tip may be parked distal to the clot. A syringe (or other pressure source) attached to the catheter is used to create negative pressure. This negative pressure draws the clot burden through the catheter and into the syringe (or other pressure source) thus extracting the unwanted clot material. The negative pressure can be applied by simply drawing back on the syringe plunger (or otherwise activating the other pressure source). An alternative practice is to simply draw back the aspiration syringe plunger against a closed stopcock, thus generating a “pre-charged” vacuum (negative pressure). Opening of the stopcock will initiate application of negative pressure to the catheter in the patient's vasculature, thus drawing the unwanted clot material into the catheter, and in turn, into the pressure source.

While this negative pressure, or “vacuum”, method has been shown to be an effective technique for drawing cloth materials out of a patient's vasculature, there are some notable drawbacks to this approach. A first drawback is the amount of force required to evacuate the syringe and lock it in a “pre-charged” vacuum state. A second drawback is the overall length of both the syringe body and the extended plunger handle.

Regarding the first problem, setting the aspiration energy with a Vac-Lock type syringe requires that the user draw back the syringe plunger over a distance of 12 cm (5 inches), while imparting a force of roughly 80 Newtons (18 lbs). In some procedures, the operator may have to apply this force 10 or more times during a procedure. This is a high force for most operators, and poses a hazard to the operator, patient, and other healthcare providers, should the syringe slip.

Regarding the second problem, the Vac-Lock type syringe, when energized for aspiration, is roughly 30 cm (12 inches) in length, which occupies considerable space on and in the operating table space. After energizing the Vac-Lock type syringe, the operator will often reposition the attached aspiration catheter, and having a roughly 30 cm appendage attached to the aspiration catheter can prove cumbersome. To address these and other issues associated with traditional Vac-Lock style syringes, the present invention provides a mechanical aspiration syringe and associated fluid flow control devices.

### BRIEF SUMMARY OF THE INVENTION

It is an objective of the present invention to provide devices that comprise mechanical aspiration syringes (and associated flow control devices) that offer, compared to traditional Vac-Lock syringes, improved mechanical advantage (providing for easier operation) with a shorter, less cumbersome physical footprint, as specified in the independent claims. Embodiments of the invention are given in the dependent claims. Embodiments of the present invention can be freely combined with each other if they are not mutually exclusive.

In some embodiments, the present invention features a mechanically actuated aspiration syringe with incorporated squeeze-type levers that work in conjunction with a ratcheting mechanism, providing a great deal of mechanical advantage as compared to traditional Vac-Lock style syringes. This allows the operator to more easily (with lower applied mechanical force) and comfortably prime the syringe to apply vacuum aspiration energy to aspirate the unwanted clot material from the patient's vasculature. Additionally, in some embodiments, the overall length of the mechanically activated aspiration syringe is shorter as compared to traditional Vac-Lock style syringes. For example, some embodiments may have a length of roughly 18 cm (7.5 inches) or even shorter, which is considerably shorter than comparable Vac-Lock style syringes, providing for less cumbersome operation when manipulating an aspiration catheter attached to the mechanically activated aspiration syringe of the present invention. Further, due to the ratcheting nature of the mechanically activated aspiration syringe, the present invention provides for the selection of a greater variety of aspiration volumes, as compared to traditional Vac-Lock style syringes.

One of the unique and inventive technical features of the present invention is two or more opposing ratchet-type handles which, when squeezed, drive a plunger of the mechanical aspiration syringe distally (thereby evacuating the syringe body). Lever handles associated with the ratchet-type handles create mechanical leverage due to their length. The force required to squeeze the ratchet levers towards each other is considerably lower than the approximately 18 lbs of force required to draw back the plunger of a Vac-Loc style syringe to an adequate aspiration volume. Furthermore, this ratcheting design allows for the ability to control the aspiration volume with much greater granularity. For example, in some embodiments, the present invention allows for the ability to control the aspiration volume from between 0 cc to 60 cc. Without wishing to limit the invention to any theory or mechanism, it is believed that the technical feature of the present invention advantageously provides for easier operation by an operator, reduced physical footprint, and greater control over aspiration volume. None of the presently known prior references or works have the unique inventive technical feature of the present invention.

Furthermore, the inventive technical feature of the present invention contributed to a surprising result. For example, the present invention, after priming the pressure source and actuating the vacuum, causes a unique flow pattern profile. For example and by way of contrast, in a typical vacuum syringe with a volume of 60 cc, the initial about 20 cc is drawn into the syringe at a vacuum pressure of about 20 mmHg, the next about 20 cc is drawn into the syringe at a vacuum pressure of about 10 mmHg, and the final about 20 cc is drawn into the syringe at a vacuum pressure of about 5 mmHg. In contrast, in the present invention, the energy stored in the tension-generating member can be tuned to provide greater initial vacuum pressure, and the pressure inside the chamber can be maintained in a more consistent manner, as more volume is displaced into the chamber. Depending on how the present invention is configured, this can result in a greater amount of aspirated material, and the ability to better withdraw adherent clot materials (e.g., those that are adhered to vessel walls).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows an isometric view of an aspiration catheter system.

FIG. 2 shows an isometric view of a vacuum-generating device of the present invention.

FIG. 3 shows a top view of a vacuum-generating device of the present invention.

FIG. 4 shows an exploded view of a vacuum-generating device of the present invention.

FIGS. 5A-5C show syringe plungers of the present invention, configured with ratchet-like teeth along their proximal body. The plunger has an elastic seal, or o-ring, to create a seal between the plunger and the syringe body. The plunger is optionally hollow and can be configured with a pin so that a tension spring can be affixed, thereby creating negative pressure when the release latch is triggered. FIG. 5A shows a side view of one such syringe plunger. FIG. 5B shows an isometric side view of one such syringe plunger. FIG. 5C shows a rear-isometric view of one such syringe plunger with a hollow plunger body and pin visible inside said hollow plunger body.

FIG. 6 shows an embodiment of a vacuum-generating device wherein the plunger is formed of transparent (e.g., plastic) material to better illustrate the function of the tension spring, which is intended to quickly draw back the plunger when activated. The quick drawback will result in negative pressure and aspiration of unwanted clot materials from the patient. The simple tension spring is used to store and release the force on the plunger.

FIGS. 7A-7D show an overview of how ratcheting of the levers of the vacuum-generating device, when squeezed and relaxed, drive the plunger in a distal direction, thereby tensioning the spring. Each squeeze of the ratchet handle positions the plunger approximately 0.5 centimeter towards the distal end of the vacuum-generating device, corresponding to an aspiration volume of approximately 5 ml. FIG. 7A shows the vacuum-generating device in its “default,” or “resting” state (i.e., where the plunger is in its natural resting position and the spring is not tensioned). FIG. 7B shows that squeezing the levers advances the plunger distally. FIG. 7C shows that subsequently relaxing the levers (after squeezing them) further drives the plunger farther distally. FIG. 7D shows the plunger advanced distally to a near-maximum extent.

FIG. 8 shows the ratcheting lever motion. When the lever is squeezed the pawl at the end of the lever engages with the pawl, thus causing the plunger to advance. In this figure the ratchet prevents the plunger from springing back, thus guiding it in a single travel direction (distally). Actuating the ratchet locking member will release the plunger and allow it to move proximally, due to the stored energy in the tension spring.

FIG. 9 shows a magnified, close-in view of the ratcheting mechanism.

FIGS. 10A-10E show the procedural steps for using the mechanical aspiration syringe of the present invention. FIG. 10A shows that prior to attaching the syringe to the aspiration catheter, the aspiration volume is set by adjusting the ratcheting levers relative to the ratchets. The spring-loaded levers are squeezed to achieve the desired aspiration volume. FIG. 10B shows the mechanical aspiration syringe set to a specific aspiration volume, here in this embodiment, the plunger being fully advanced to an aspiration volume of 60 cc. FIG. 10C shows the mechanical aspiration syringe connected to the aspiration catheter. After the syringe is attached to the aspiration catheter, the stopcock can be placed in the open position to prepare for aspiration of clot material. FIG. 10D shows that when the ratchet locking member is pressed, the plunger is released, initiating aspiration. FIG. 10E shows the final steps of operation, which involves closing the stopcock and detaching the syringe. If warranted, the aspirated blood and clot material can optionally be deposited into a filter device (as pictured), or discarded, in either case by squeezing the ratcheting levers to advance the plunger, therefore discharging the syringe's contents.

FIG. 11 shows a comparison of the induced velocity profile (i.e., flow profile through the catheter) of the present invention (referred to here as “Mechanical” and indicated by the line with data points represented by circles) compared to a traditional Vac-Lock syringe (referred to here as “Vac-Lock” and indicated by the line with data points represented by squares). The induced velocity profile of the two syringe types can differ considerably, and may be correlated with overall aspiration performance as measured by one or more of the following parameters: total clot volume, instances of catheter clogging, positional distance allowance between the catheter and clot material, and other factors. The tension spring of the present invention may be tuned to change and improve the induced velocity profile (i.e., flow profile through the catheter).

FIGS. 12A-12D show that, when high flow rates of blood travel through the syringe (or other pressure source) and impact the syringe plunger face (or other surface) at high velocity, the high impact velocity that results can cause damage to the blood cells in the blood, and also show a fluid dampening shuttle valve (i.e., “decoupling damper”) to reduce said damage. The high velocity of impact can cause, for example, mechanical hemolysis, wherein said blood cells are deformed and/or ruptured due to the high impact forces, thereby causing the release of hemoglobin from said ruptured cells. If the blood is to be reinfused into the patient, this degradation in blood quality can be detrimental. FIG. 12A shows that blood may move proximally through the chamber, towards the face of the plunger. FIG. 12B shows that blood may impact the plunger face at high velocities, causing damage to blood cells. FIG. 12C shows a fluid dampening shuttle valve (i.e., “decoupling damper”), comprising a dampening spring. The decoupling damper provides multiple advantages. First, the stream of blood entering the syringe body (or other pressure source) will be evenly distributed on the face of the decoupling damper, thus preventing the blood from splattering randomly into an open syringe body, leading to foaming (aeration) of the blood. Fluid velocity at the distal tip of the aspiration catheter, however, is unaffected by the decoupling damper. Second, when the decoupling damper reaches the face of the syringe plunger, the spring integrated into the decoupling damper dampens the final impact, preventing damage to viable blood cells. FIG. 12D shows compression of the dampening spring upon impact of the decoupling damper with the plunger face.

FIGS. 13A-13C show procedural steps of the decoupling damper in use. FIG. 13A shows step 1 and 2. Step 1 involves attaching the aspiration syringe (or other pressure source) to the aspiration catheter. Step 2 involves pulling the plunger of the syringe into its fixed position (on a locking syringe; or otherwise activating or priming the pressure source). In the case of a 60 cc locking syringe, ˜24-29 inHg of pressure will be generated in the syringe body. The decoupling damper will stay at the distal position as the closed stopcock (or other fluid control device) prevents differential pressure from activating the damper. FIG. 13B shows step 3. In step 3, the stopcock is opened. Blood rushes into the syringe body (or other pressure source), causing the decoupling damper to collimate the fluid and travel to the syringe plunger. FIG. 13C shows steps 4 and 5 and optional step 6. In step 4, the stopcock (or other fluid control device) is closed. In step 5, the syringe (or other pressure source) is detached from the aspiration catheter for disposal or filtration. Optional filtration prior to optimal reperfusion is shown as an optional step 6 at the bottom of FIG. 13C.

FIG. 14 shows how a slit valve may be used to prevent fluid leakage when the syringe (or other pressure source) is disconnected from the aspiration catheter.

FIG. 15 shows the syringe (or other pressure source) being pressed through the cross slit in the elastic material that, in this embodiment, forms the slit valve, thus providing an open path for fluid aspiration. The syringe (or other pressure source) tip may be pressed into the slit valve, opening the slit in the (in this embodiment, elastic) seal of the slit valve, allowing the syringe (or other pressure source) to come into fluid communication with the patient's blood inside of the aspiration catheter.

FIGS. 16A-16C shows a duck bill valve. FIG. 16A shows the duck bill valve on the aspiration port, disconnected from the pressure source (here, a syringe). The syringe (or other pressure source) can be disconnected from the duckbill valve via a push lever latch (as pictured) or similar mechanism. FIG. 16B shows the duck bill valve on the aspiration port connected to the syringe. FIG. 16C shows an interior view of the duck bill valve.

FIGS. 17A-17C shows alternative embodiments of duck bill valves. FIG. 17A shows a certain embodiment of a duck bill valve from three different perspectives. FIG. 17B shows another alternative embodiment of a duck bill valve. FIG. 17C shows yet another alternative embodiment of a duck bill valve.

FIGS. 18A-18C show the fluid de-coupling mechanism of the duck bill valve. FIG. 18A shows an elongated syringe tip can be used to mate with the duck-bill valve. The syringe tip may be optionally secured to the valve housing (housing the duck bill valve) via a latching connector. FIG. 18B shows a partially transparent valve housing and duck bill valve, wherein the elongated syringe can be seen crossing into the duck bill valve. FIG. 18C shows an exploded view of the valve housing, duck bill valve, and optional latch or other mechanism for securing the syringe (or other pressure source) to the valve housing.

FIGS. 19A-19B show a “gate” valve (i.e., a “slider” valve). FIG. 19A shows an exploded view of a gate valve. FIG. 19B shows a partial cutaway section view of a gate valve.

FIGS. 20A-20B show another embodiment of a gate valve. FIG. 20A shows an isometric view of a gate valve. FIG. 20B shows an alternative view of a gate valve.

FIGS. 21A-21C show an alternative “rotational flat” style gate valve. FIG. 21A shows an isometric view of a rotational, flat style gate valve. FIG. 21B shows a partially-transparent view of a rotational flat style gate valve with the gate in its “closed” position. FIG. 21C shows a partially-transparent view of a rotational flat style gate valve with the gate in its “open” position.

FIGS. 22A-22D show an alternative “low profile” embodiment of a “rotational flat” style gate valve. This embodiment has a smaller profile than most other gate valves disclosed herein, and also has an optional lever, with the lever's position optimized for single-handed operation. The lever may operate whether the gate is “lowered” or “raised.” FIG. 22A shows an isometric view of a low profile rotational flat style gate valve with a lever. FIG. 22B shows a front view of a low profile rotational flat style gate valve with a lever. FIG. 22C shows a rear view of a low profile rotational flat style gate valve with a lever. FIG. 22D shows a rear view of a low profile rotational flat style gate valve with a lever, with the gate in a semi-open, semi-closed intermediate position. The smaller o-ring (located closer to the center of the flow path, as defined by the ports in the gate valve) provides a seal when the gate is perfectly aligned in either the “open” or “closed” position. A second, larger o-ring (located closer to the periphery of the flow path) prevents fluid leakage while the gate is “swung” from “open” to “closed” positions (and vice-versa). In FIG. 22D, the gate is in an intermediate position (between “open” and “closed”) where, but for the second, larger o-ring, fluid could leak out from the gate valve during “swing” of the gate.

FIGS. 23A-23C show alternative embodiments of gate valves. FIG. 23A shows a first set of gate valves with a traditional lever design, the lever used to move the gate valve from the “open” to “closed” position, and vice versa. FIG. 23B shows alternative embodiments of gate valves with “slider” style levers. Slider style levers have the advantage of being lower profile than “traditional” lever designs. FIG. 23C shows alternative embodiments of gate valves with slider style levers.

FIG. 24A-24B show plunger valves for thrombectomy. FIG. 24A shows a plunger valve in the “closed” position. FIG. 24B shows a plunger valve in the “open” position. Note that the rotatable knob attached to the plunger has been rotated. Ideally, a plunger valve should meet a number of design requirements, including: being easy to use (e.g., requiring only a one-quarter to three-quarter turn to actuate the plunger; having a barbed inlet/outlet compatible with aspiration tubing; having an overall size comparable with valves of other style, allowing plunger valves to fit into existing packaging; having a clear housing to visualize blood or other contents with the plunger valve body, etc.) Plunger valves may also optionally have minimum flow restrictions. In some embodiments, minimum flow restrictions are >0.280 inches in diameter. The valves may be sterilized through various means, including the use of ethylene oxide (ETO). In some embodiments, plunger valves of the present invention may have an aging temperature of 55 degrees celsius for 111 days. In some embodiments, plunger valves of the present invention have pressure ratings of positive pressures of >50 PSI and negative pressures of >22 mmHg vacuum. In some embodiments, the plunger valves are characterized by positional stability (i.e., when the valve is open it stays open, and when the valve is shut, it stays shut). In some embodiments, the plunger valves may have limit stops (i.e., mechanical stops) or other override prevention features that prevent a user from damaging the valve.

FIG. 25 shows a partial cutaway view of a plunger valve.

FIG. 26 shows an exploded view of a plunger valve.

FIGS. 27A-27C show a plunger valve design with o-ring(s). FIG. 27A shows the plunger valve in a closed position. The lower o-ring is configured to seal off the two ports (left and right) disposed on the valve body when the plunger is in the “closed” position. FIG. 27B shows the plunger valve in an open position. The lower o-ring fluidically couples the two ports when the plunger is in the “opened” position. FIG. 27C shows the plunger valve in an “intermediate” position, as occurs when moving the plunger from the “open” position to the “closed” position (or vice-versa). When moving the plunger between “opened” and “closed,” leakage may occur around the bottom o-ring, potentially causing fluid to flow up the body of the plunger, towards the valve stem. In these instances, the top o-ring prevents said leakage from spreading farther up the plunger.

FIGS. 28A-28B show one embodiment of a plunger valve of the present invention. FIG. 28A shows an assembled plunger valve, as optionally attached to a catheter equipped with a stopcock. FIG. 28B shows a partially disassembled plunger valve.

FIGS. 29A-29B show an alternative embodiment of a plunger valve equipped with threading. FIG. 29A shows an exploded view of a plunger valve with threading. FIG. 29B shows an assembled plunger valve with threading, with the knob shown as partially transparent to allow visualization of the knob's internal threading.

FIGS. 30A-30B show a threaded plunger valve. FIG. 30A shows how manipulation of the knob moves the plunger between the closed and open positions. FIG. 30B shows assembly steps of the threaded plunger valve. In step 1, the threaded plunger valve is disassembled. In step 2, the bottom cap is placed around the valve body. In step 3, the threaded knob is placed over the valve body, on top of the bottom cap. In step 4, the threaded knob is bonded to the bottom cap.

FIGS. 31A-31B show an embodiment of a plunger valve with alternative actuation mechanisms. FIG. 31A shows a plunger valve with a finger “slider switch” that may be used to move the plunger from the “open” to “closed” position and vice-versa. FIG. 31B shows a plunger valve with a “toggle switch” that may be used to move the plunger from the “open” to “closed” position and vice-versa.

FIG. 32 shows plunger valves with alternative actuation handle designs (designed to replace a knob) focused on ease of use, ergonomics, and size.

FIGS. 33A-33C show an alternative “push-pull” valve actuation mechanism for plunger valves. In these embodiments, the plunger is actuated by pushing and/or pulling the plunger via either the plunger itself (with one end of the plunger protruding from the valve body, and thus manipulable by an operator) or a linearly-actuated handle disposed on one end of the plunger, with the handle disposed coaxially with the plunger. FIG. 33A shows a plunger valve with a push-pull mechanism in the closed position. FIG. 33B shows a plunger valve with a push-pull mechanism in the open position. FIG. 33C shows a cross-section view of a plunger valve with a push-pull mechanism. Note that the valve body may be optionally configured with a restraint edge or “lip” that prevents the push-pull plunger from separating from the valve body.

FIGS. 34A-34B show an embodiment of a normally closed fluid control device, in this case, a normally closed plunger valve. FIG. 34A shows the normally closed plunger valve with the plunger in its default, closed position. FIG. 34B shows the normally closed plunger valve with the plunger in its open position. In this embodiment, the normally closed plunger valve is held in its normally closed position via spring tension. To open the valve, an operator may pull on the plunger handle to overcome the spring tension and move the plunger in its open position.

FIGS. 35A-35B show an alternative embodiment of a normally closed fluid control device, in this case, a normally closed plunger valve operated by a cam lever. The cam lever can be actuated by an operator to move the plunger from its default “closed” state (as achieved by spring tension) to its open state. FIG. 35A shows the normally closed plunger valve with cam lever with its plunger in its default, closed position. FIG. 35B shows the same plunger valve with its cam lever actuated, thereby moving the plunger into its open position.

### DETAILED DESCRIPTION OF THE INVENTION

Following is a list of elements corresponding to a particular element referred to herein:
010 Vacuum-generating device
025 Catheter
050 Fluid control device
075 Hollow member
100 Chamber
101 Proximal chamber end
102 Distal chamber end
120 Aspiration port
199 End cap
200 Vacuum plunger
201 Proximal plunger end
202 Distal plunger end
203 Plunger outer circumference
210 Pawl
300 Tension-generating member
301 Tension-generating member proximal end
302 Tension-generating member distal end
310 Tension-generating member attachment point
400 Ratcheting lever
401 Proximal ratcheting lever end
402 Distal ratcheting lever end
500 Ratchet locking member
600 Fluid decoupling damper
601 Fluid decoupling damper proximal end
602 Fluid decoupling damper distal end
610 Energy-absorbing member
700 Slit valve assembly
710 Seal housing
711 Proximal housing end
712 Distal housing end
715 Seal receptacle
721 Proximal housing port
722 Distal housing port
730 Elastomeric seal
731 Proximal elastomeric seal side
732 Distal elastomeric seal side
735 Slit
750 Slit valve lever
774 Tapered structure
775 Elongated tip
800 Gate valve assembly
810 Valve bonnet
811 Valve bonnet opening
820 Valve seat
821 Valve seat opening
830 Valve gate
831 Valve gate opening
835 Valve gate lever
836 Low-profile switch
837 Valve gate hinge
900 Plunger valve assembly
910 Valve body
911 Valve cavity
912 Plunger seat
915 Plunger valve port
920 Valve plunger
921 Distal terminus of valve plunger
925 Spring
930 Valve plunger actuator
931 Rotatable knob
932 Low-profile slider switch
933 Toggle switch
934 Linearly-actuated handle
935 Cam lever
939 Knob bottom cap
950 Pin
951 Pin groove

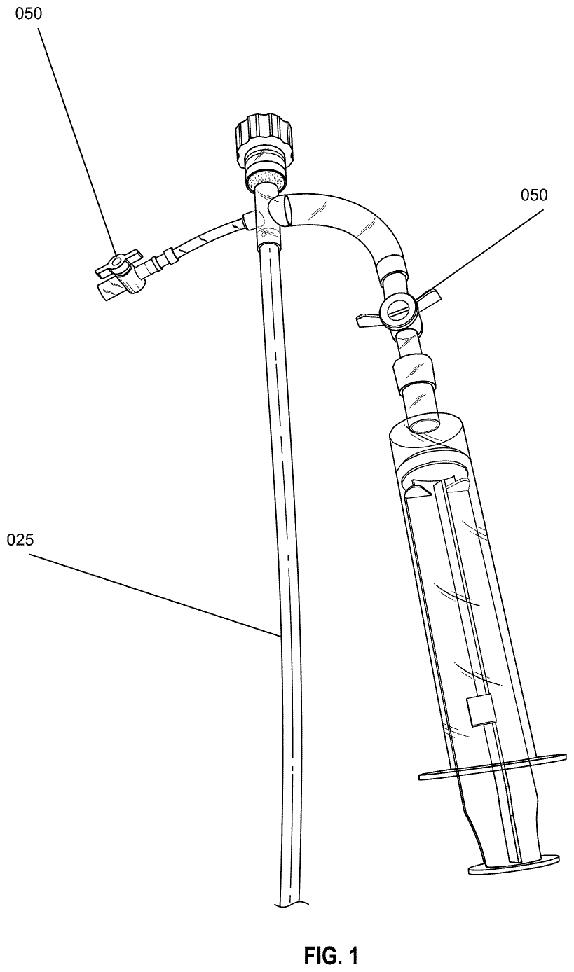

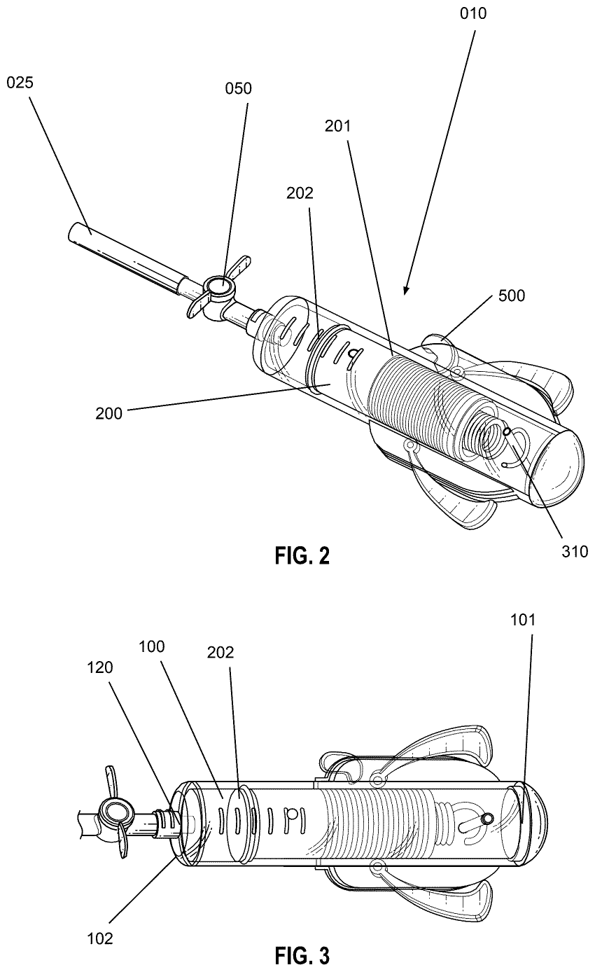

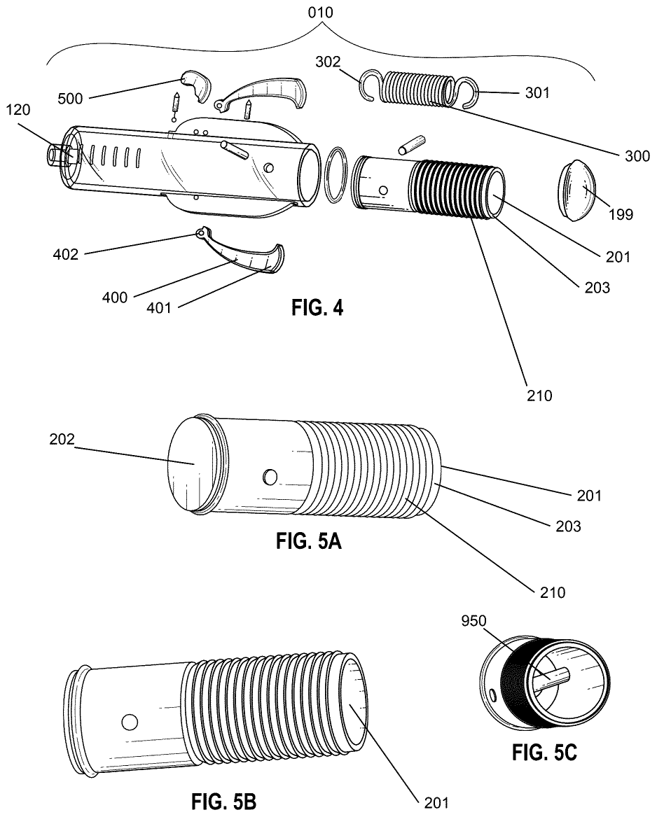

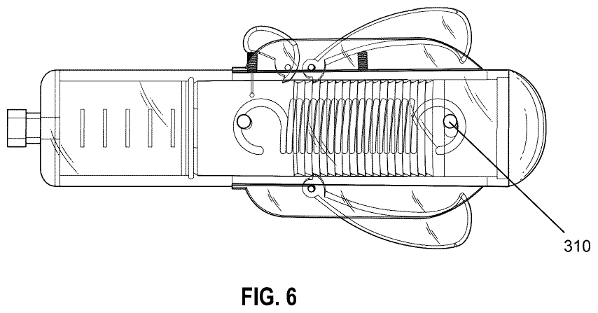

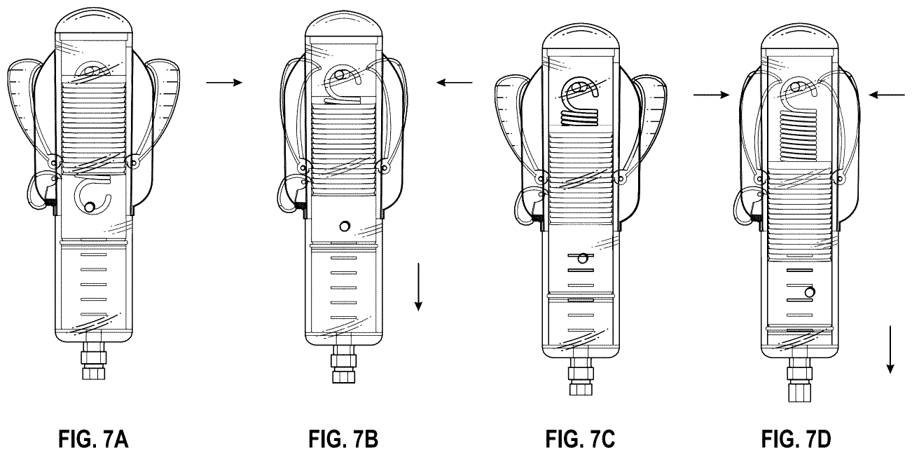

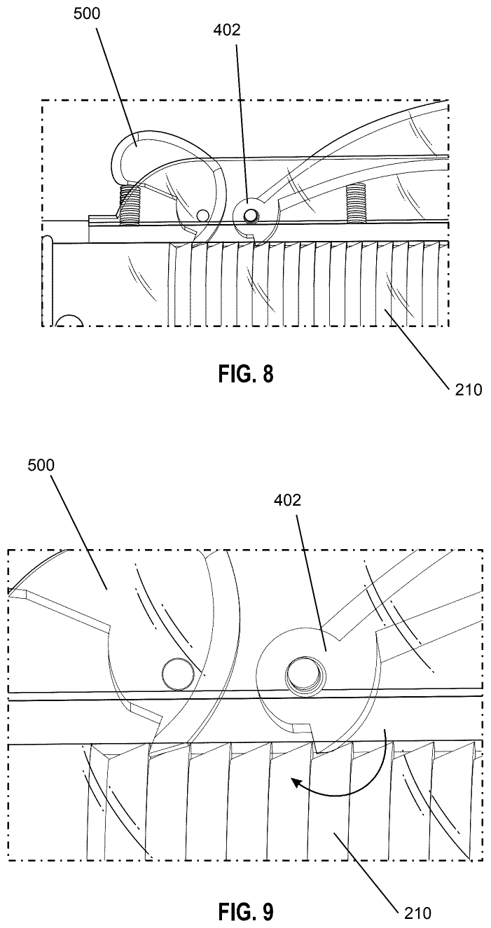

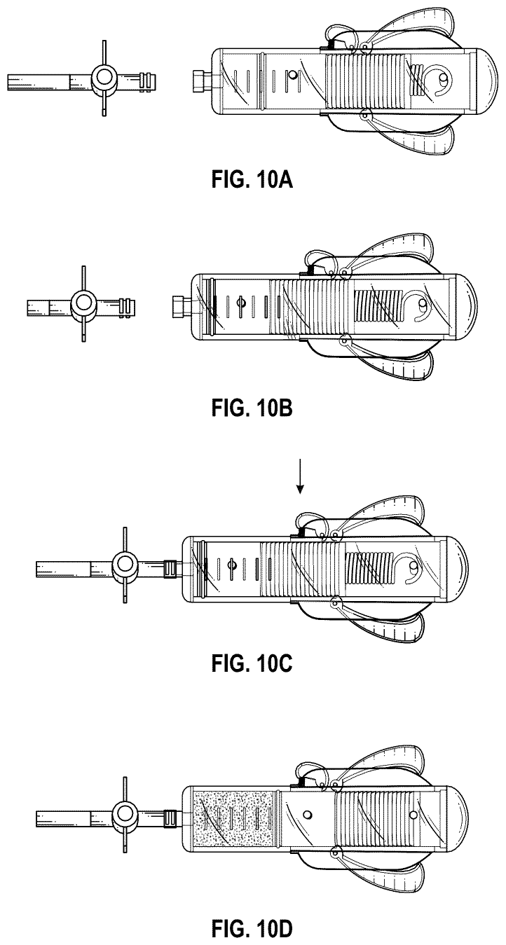

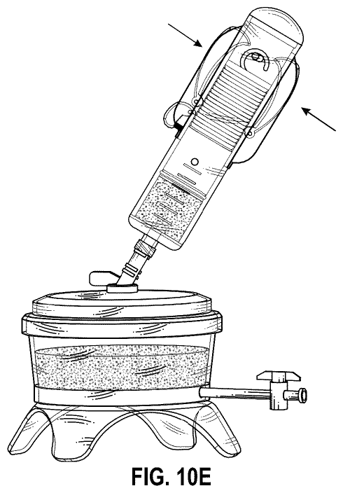

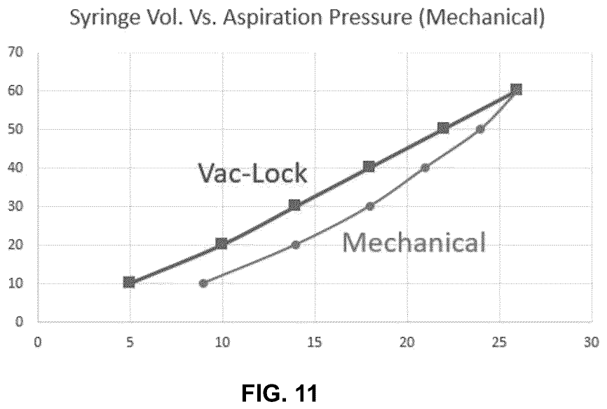

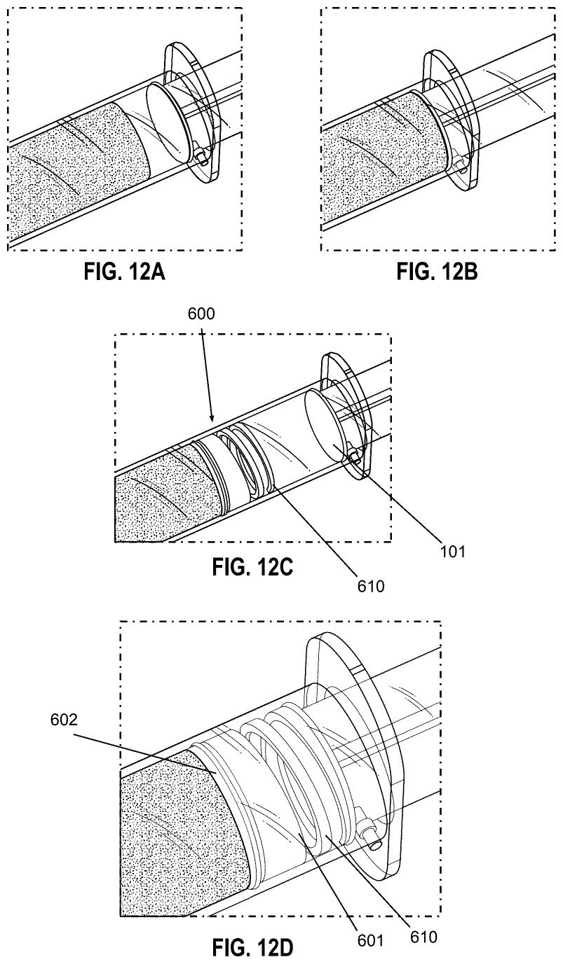

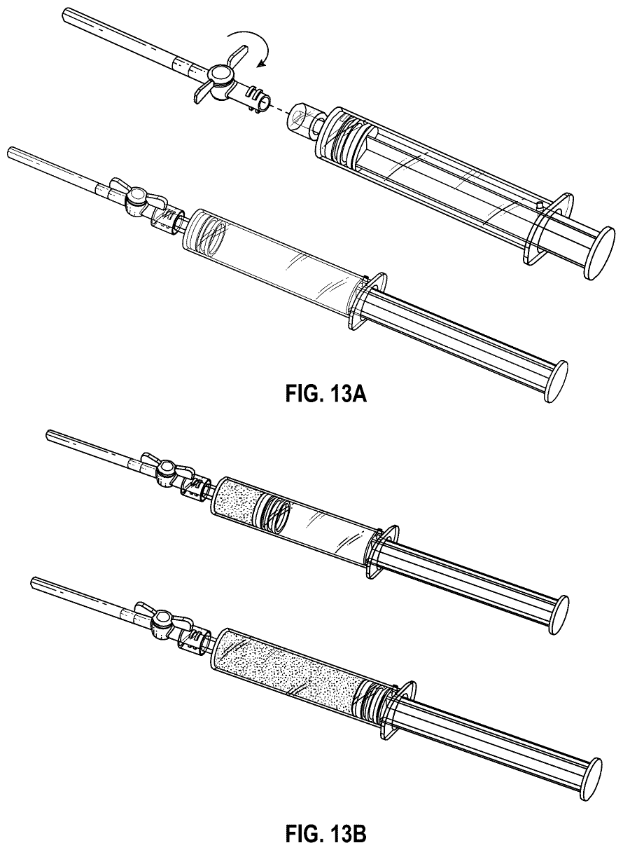

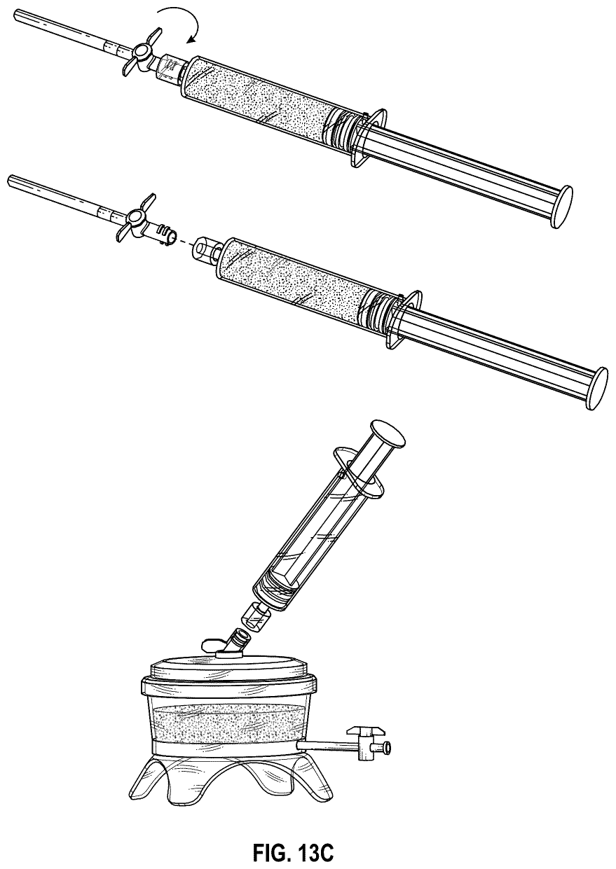

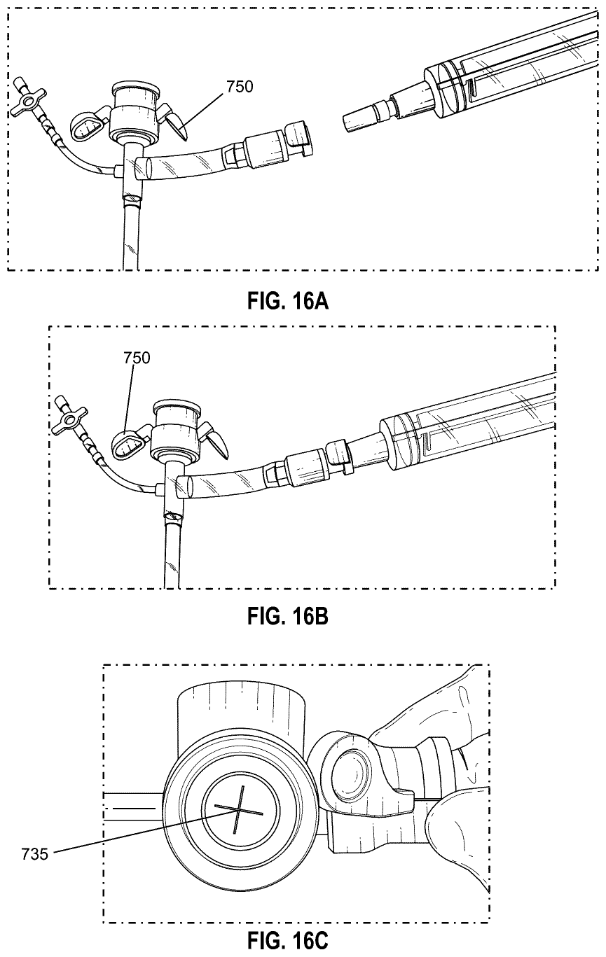

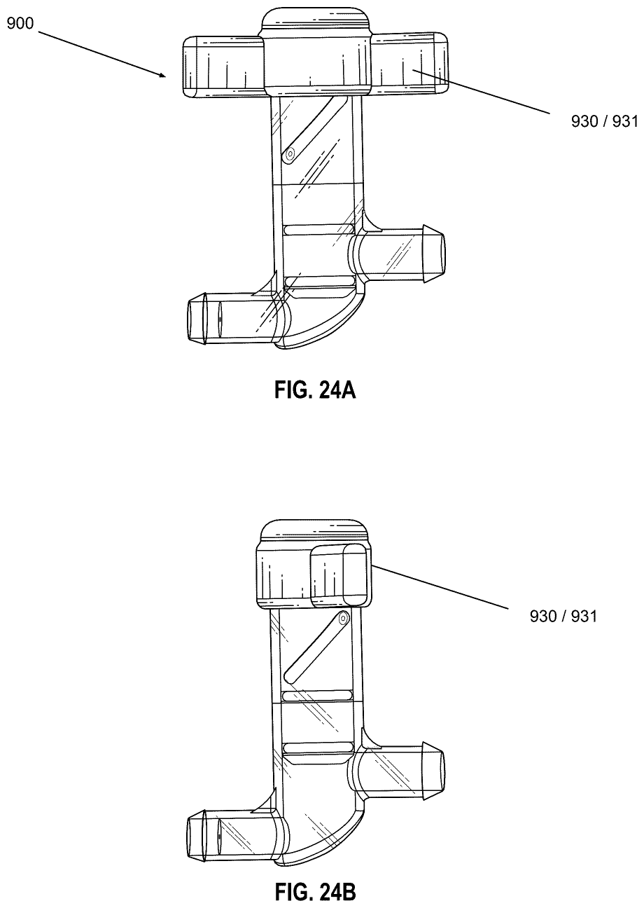

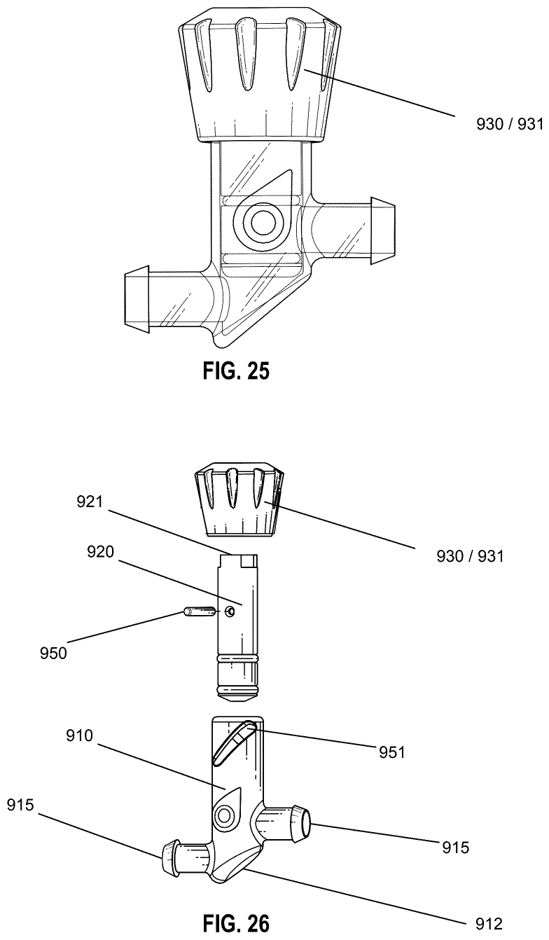

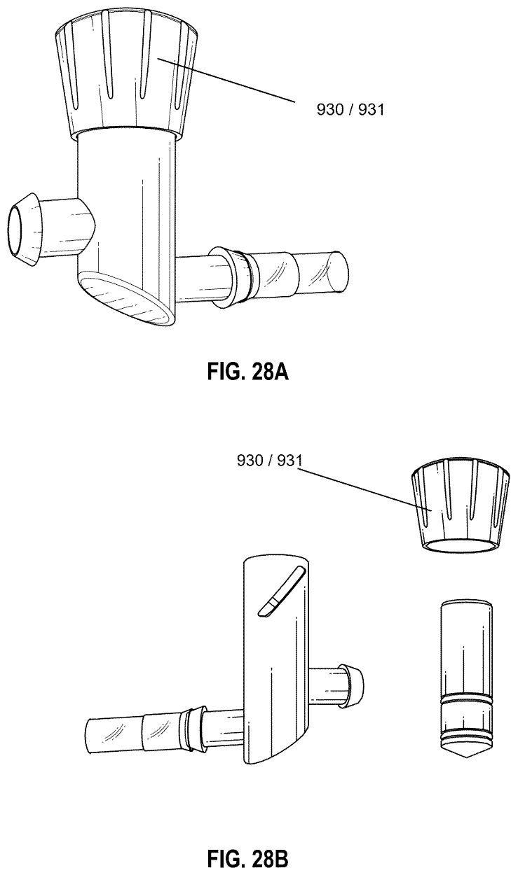

Referring now to FIGS. 1-35B, the present invention features a vacuum-generating device (010) for intravascular aspiration of clot material, the device comprising: a plunger (200) having a proximal plunger end (201) and a distal plunger end (202), the plunger (200) comprising one or more pawls (210) disposed at least partially around an outer circumference (203) of the plunger (200); a chamber (100) having a proximal chamber end (101) and a distal chamber end (102), the plunger (200) slidably disposed within the chamber (100) and configured to form a fluid-tight seal with an inner wall of the chamber, the chamber (100) configured to generate a vacuum when the plunger (200) is drawn towards the proximal chamber end (101), the distal chamber end (102) having an aspiration port (120) disposed thereon; a tension-generating member (300) having a proximal end (301) and a distal end (302), disposed within the chamber (100), the distal tension-generating member end (302) operatively connected to the proximal plunger end (201), the proximal tension-generating member end (301) operatively connected to a tension-generating member attachment point (310), the tension-generating member attachment point (310) disposed on or proximate to the proximal chamber end (101); the tension-generating member (300) configured to apply a force to the plunger (200) and draw the plunger (200) towards the proximal chamber end (101); a ratcheting lever (400) having a proximal ratcheting lever end (401) and a distal ratcheting lever end (402), the distal ratcheting lever end (402) configured to engage the one or more pawls (210) of the plunger (200), the proximal ratcheting lever end (401) configured to move the distal ratcheting lever end (402) along the pawls (210) towards the proximal plunger end (201) when the proximal ratcheting lever end (401) is actuated; and a ratchet locking member (500) configured to engage the one or more pawls (210), selectively hold the plunger (200) in a tensioned position, and release the plunger upon actuation of the ratchet locking member (500); wherein the plunger (200), when drawn towards the distal chamber end (102), generates potential energy in the tension-generating member (300); wherein a vacuum is generated and released via the aspiration port (120) thereby aspirating at least a portion of the clot material into the chamber (100) when the ratchet locking member (500) is actuated when the plunger (200) is in a tensioned position, thereby releasing the plunger (200) from its tensioned position and allowing it to travel towards the proximal chamber end (101), thereby generating a vacuum.

In some embodiments, the vacuum-generating device further comprises an end cap (199) disposed on the proximal end (101) of the chamber (100).

In some embodiments, the vacuum-generating device further comprises a fluid control device (050) disposed distally to the distal chamber end (102) and fluidly connected to the chamber (100) via the aspiration port (120). In some embodiments the fluid control device (050) is a check valve or one-way valve (e.g., duckbill valve, flapper valve, umbrella valve, ball-and-spring check valve, diaphragm check valve, slit valve or cross-slit valve, etc.) stopcock or directional valve (e.g., 3-way stopcock, 4-way stopcock, rotary manifold valve, etc.) pressure-regulating valve (e.g., relief valve or safety valve, pressure-limiting valve, cracking-pressure valve), shutoff/isolate valve (gate valve, ball valve, buttery valve, poppet valve, slide valve) flow-control valve (e.g., needle valve, pinch valve, roller clamp, proportional solenoid valve), rotating valve, plunger valve). In some embodiments the fluid control device (050) is an electrically actuated valve (e.g., solenoid valve, piezoelectric valve, motorized ball/rotary valve).

In some embodiments, the present invention features a fluid decoupling damper (600) for use in a chamber (100) having a proximal chamber end (101) and a distal chamber end (102), the decoupling damper (600) having a proximal end (601) and a distal end (602), the decoupling damper (600) further comprising an energy-absorbing member (610) disposed on the decoupling damper (600) proximal end (601), the energy-absorbing member (610) may be configured to, upon aspiration of fluid into the chamber (100), at least partially absorb impact forces imparted to the decoupling damper (600) from the decoupling damper (600) making contact with the proximal end (101) of the chamber (100). In some embodiments, the energy-absorbing member (610) is a spring, an elastomeric/flexible block or bumper, foam absorber, flexure element or compliant element, bellows, hydraulic damper, fluid-filled sac or bladder, gas-filled sac or bladder, air chamber/pneumatic cushion, air piston, compressible bladder, sliding friction pad, crush tube or cone, honeycomb structure, viscoelastic polymer, composite layup, gel-based absorber, deformable member, etc.

In some embodiments, the present invention features a slit valve assembly (700) for use in intravascular thrombectomy, comprising: a seal housing (710) comprising a seal receptacle (715) disposed within the seal housing (710), a proximal housing end (711), a distal housing end (712), a proximal housing port (721) disposed on the proximal housing end (711), and a distal housing port (722) disposed on the distal housing end (712), the proximal housing port (721) fluidly connected to the seal receptacle (715), the seal receptacle (715) fluidly connected to the distal housing port (722), the distal housing port fluidly connected to a catheter (025); an elastomeric seal (730) disposed in the seal receptacle (715), the elastomeric seal (730) having a slit (735) fluidly connecting a proximal elastomeric seal side (731) to a distal elastomeric seal side (732); wherein, the elastomeric seal (730) is self-sealing when fluid is present on the distal elastomeric seal side (732) such that said fluid will not substantially leak from the distal elastomeric seal side (732) of the slit (735) onto the proximal elastomeric seal side (731); wherein, when a hollow member (075) is inserted from the proximal elastomeric seal side (731) through the slit (735), fluid may flow from the catheter (025), through the slit (735), and into the hollow member (075).

In some embodiments the seal housing (710) is formed from Polyether ether ketone (PEEK), Polyphenylsulfone (PPSU), Polytetrafluoroethylene (PTFE), polypropylene, high density polyethylene (HDPE), medical grade polycarbonate, Ultra-High Molecular Weight Polyethylene (UHMWPE), or a similarly suitable material. In some embodiments the elastomeric seal (730) is formed from medical-grade platinum-cured silicone/vinyl methyl silicone rubber (VMQ), medical thermoplastic elastomer (TPE), medical-grade thermoplastic polyurethane (TPU), fluoroelastomers (e.g., FKM, FFKM), nitrile (NBR), or a similarly suitable material.

In some embodiments, the elastomeric seal (730) is operatively connected to one or more slit valve levers (750) and is configured to force the slit (735) into an open configuration when the one or more slit valve levers (750) are actuated by an operator. In some embodiments, the slit (735) returns to a closed configuration when the one or more slit valve levers (750) are released by the operator.

In some embodiments, the elastomeric seal (730) forms a tapered structure (774) that diverges proximally from the distal elastomeric seal side (732) and converges distally to the distal elastomeric seal side (732) to form an elongated tip (775) disposed distal to the distal elastomeric seal side (732), wherein one or more slit (735) is formed at the distal end of the elongated tip (775), and wherein the elongated tip (775) is symmetrical along at least one of its bilateral axes, i.e., a duckbill valve.

In some embodiments, the present invention features a gate valve assembly (800) for thrombectomy comprising: a valve bonnet (810) having a valve bonnet opening (811); a valve seat (820) having a valve seat opening (821); and a valve gate (830) having a valve gate opening (831), the valve gate (830) configured to slidably fit between the valve bonnet (810) and valve seat (820); wherein the valve gate (830) may be slidably positioned relative to the valve bonnet (810) and valve seat (820); wherein, when the valve gate (830) is positioned such that valve gate opening (831) is substantially aligned with the valve bonnet opening (811) and valve seat opening (821), the valve bonnet opening (811), valve gate opening (831), and valve seat opening (821) are in fluid communication; and wherein, when the valve gate (830) is positioned such that the valve gate opening (831), valve bonnet opening (811), and valve seat opening (821) are not substantially aligned, at least two of the valve gate (830), valve bonnet opening (811), and valve seat opening (821) are fluidically disconnected.

In some embodiments, the valve gate (830) is operably connected to a valve gate lever (835), wherein the valve gate lever (835) is configured to slidably move the valve gate (830) relative to the valve bonnet (810) and valve seat (820) when the valve gate lever (835) is actuated.

In some embodiments, the valve gate (830) is rotationally slidable around a valve gate hinge (837), the valve gate hinge (837) operably connecting the valve gate (830) to at least one of the valve bonnet (810) or valve seat (820). In some embodiments, the valve gate (830) is operably rotatable around the valve gate hinge (837) via actuation of a valve gate lever (835). In some embodiments, the valve gate lever (835) comprises a low-profile switch (836).

In some embodiments, the present invention features a plunger valve assembly (900) for use in intravascular thrombectomy comprising: a valve body (910) comprising an valve cavity (911) disposed within the valve body (910), a plunger seat (912) disposed at the proximal terminus of the valve cavity (911), and at least two plunger valve ports (915) fluidly connected to the valve cavity (911); a valve plunger (920) configured to slidably fit within the valve cavity (911); a valve plunger actuator (930) disposed at the distal terminus of the valve plunger (921) and configured to, upon actuation, move the valve plunger (920) between a first valve plunger position and a second valve plunger position; wherein, the valve plunger (920) fluidically disconnects the at least two plunger valve ports (915) from each other when in the first plunger position; wherein the at least two plunger valve ports (915) are fluidically connected when the plunger valve is in the second plunger position.

In some embodiments, the valve body (910) and/or valve plunger (920) may be formed from stainless steel (e.g., 316L stainless steel), an titanium alloy (e.g., Ti-6Al-4V), Polyether ether ketone (PEEK), Polyphenylsulfone (PPSU), Polytetrafluoroethylene (PTFE), polypropylene, high density polyethylene (HDPE), medical grade polycarbonate, Ultra-High Molecular Weight Polyethylene (UHMWPE), medical grade thermoplastic polyurethane (TPU), or a similarly suitable material.

In some embodiments, the valve plunger actuator (930) comprises a rotatable knob (931). In some embodiments, the valve plunger actuator (930) comprises a low-profile slider switch (932). In some embodiments, the valve plunger actuator (930) comprises a toggle switch (933). In some embodiments, the valve plunger actuator (930) comprises a linearly-actuated handle (934). In some embodiments, the valve plunger actuator (930) comprises a cam lever (935).

In some embodiments, the plunger body (910) further comprises a pin groove (951), and the valve plunger (920) further comprises a pin (950), wherein the pin (950) is configured to slidably fit within the pin groove (951). In some embodiments, the valve plunger (920) comprises a normally-closed plunger, wherein the valve plunger (920) is held in its normally closed position via spring tension provided by a spring (925) or similar mechanism.

In some embodiments, the present invention uses a stopcock or similar valve or fluid control device to control the flow of fluid, e.g., to prevent fluid from flowing out of the catheter when an aspiration syringe is detached from the aspiration catheter. In some embodiments, the stopcock is a large bore stopcock (as used herein, the term “large bore” refers to stopcocks with flow path diameters of 0.2 inches or greater).

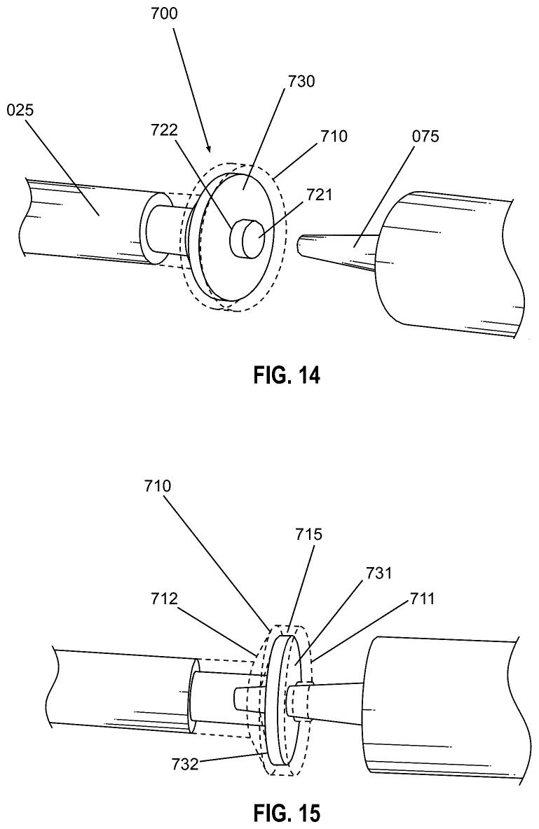

In other embodiments, the stopcock may be substituted (or supplemented) with an alternative closure mechanism. In some embodiments, alternative closure mechanisms may include, but are not limited to, slit valve(s) (see, for example, FIGS. 14-18C). One advantage of using a slit valve is that when the syringe is pulled from the catheter (i.e., when the syringe and catheter are decoupled), the slit valve will close itself, preventing fluid from spilling out of the catheter. Furthermore, depending on the configuration of the device generating vacuum pressure (e.g., syringe), aspiration may also be initiated by docking said device with the slit valve on the aspiration catheter (i.e., aspiration may be initiated by coupling the device generating vacuum pressure (e.g., syringe) and the catheter, by crossing the slit valve/duck bill valve (as discussed later herein) with the device generating vacuum pressure). The slit valve may, in some embodiments, be formed from a compliant material. In some embodiments, the slit valve is formed from an elastomeric material. In some embodiments, the slit valve has two center slits, placed under slight compression by the slit valve's housing. In some embodiments, the slit valve is capable of preventing leakage from the catheter upon disconnection of the syringe (or other pressure source) while under typical vascular pressure (about 2 PSI or less).

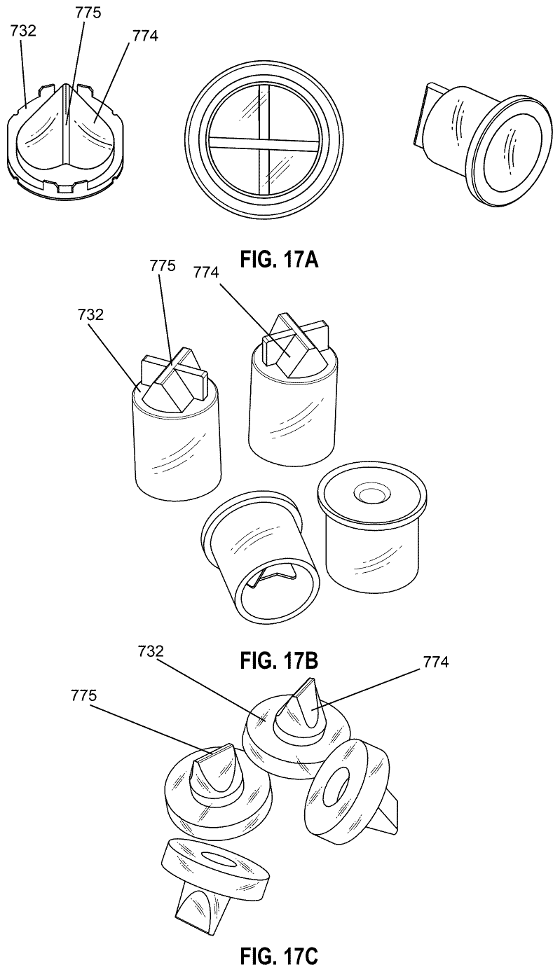

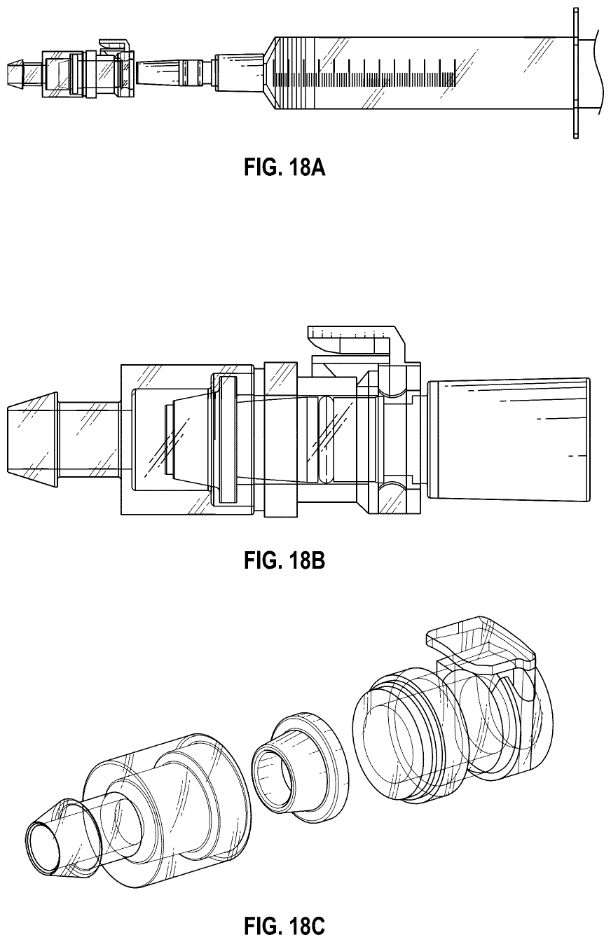

In other embodiments, alternative closure mechanisms may include duck bill valves (see, for example, FIG. 17A-18C). The duck bill valve (i.e., a tapered structure that diverges proximally from the distal elastomeric seal side and converges distal to the distal elastomeric seal side to form an elongated tip, wherein the slit(s) is/are formed at the distal end of the elongated tip, and wherein the elongated tip is symmetrical along at least one of its bilateral axes) operates on a similar principle as the slit valve, in that both rely on the elastic properties of the materials from which they are constructed, and both are self-sealing and prevent fluid from spilling out of the catheter upon disconnection of the syringe (or other pressure source). However, the duck bill valve benefits from additional pressure resistance due to the geometry of the elastomeric valve itself. The duck bill valve may take on a variety of configurations and sizes.

In still other embodiments, alternative closure mechanisms may include alternative valve designs. By way of background, traditional stopcock valves used in medical devices achieve fluid control by way of balanced interference between the valve body and valve seal. An “interference fit” requires a slightly larger and slightly smaller component, one relatively soft and one relatively hard, thereby creating a leak free “interference fit” seal when the two components are mated together. However, to achieve leak free performance, a balance must be struck between the following parameters: torque resistance, temperature stability, relative material properties, extremely tight tolerances (to maintain a precise interface fit between components, for example, the valve body and valve stem), dimensional repeatability (in molded part processing), molding tooling, and valve geometry. Attempts to develop large bore stopcocks have been met with challenges related to the requirement of very tight tolerances. This is particularly true as the bore is increased beyond 0.2 inches. Thus, the aforementioned “balance” of design elements is difficult to achieve, most notably due to molding- and manufacturing-related challenges. Alternative valve designs of the present invention help to overcome these issues. Some alternative valve designs disclosed herein achieve leak-free performance partially by incorporation of elastomeric o-rings, and take the form of slider valves (i.e., gate valves) for thrombectomy and plunger valves for thrombectomy.

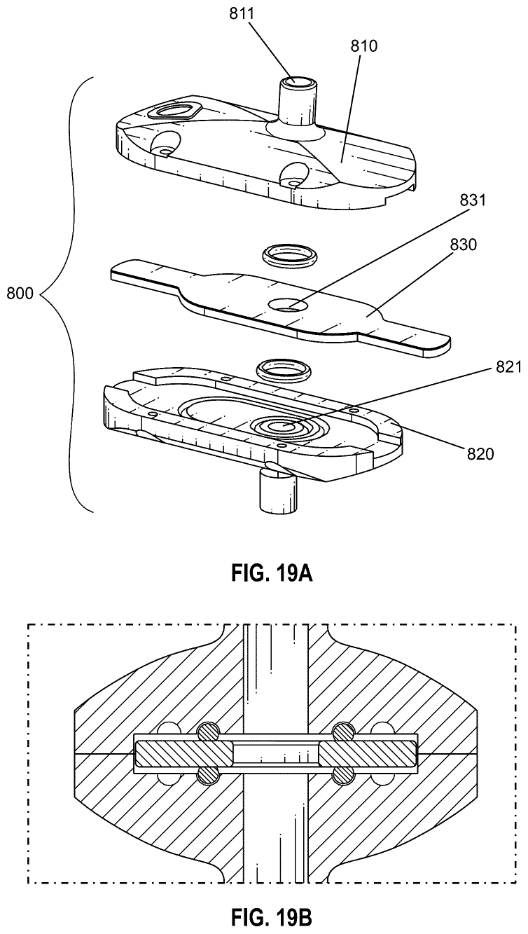

In some alternative valve designs, the present invention features a gate valve for thrombectomy (i.e., a slider valve for thrombectomy) (see, for example, FIGS. 19A-23C). In some embodiments, the gate valve operates off the principle of moving a gate's (i.e., slider's) position relative to ports within a valve body to thereby control fluid flow. When the gate (i.e., slider) is “lifted” it is positioned out of the flow path (defined by the position of the port(s) within the valve body) thereby allowing flow of fluid through the valve body. When the gate is “lowered” it is positioned within the flow path, blocking the flow of fluid through the valve body. In some embodiments, gate valves may have a plurality of o-rings. O-rings, optionally made of elastomeric material, may be optionally used to provide a seal between the valve body and the gate(s). The O-ring may be formed of a material with a desirable level of compliance, for example, an elastomeric material. Selection of an o-ring with an appropriate level of compliance allows for the o-ring to compensate for manufacturing variances in the gate/slider and valve body, including but not limited to to account for and allow for thermal expansion of the gate/slider and/or valve body. In some embodiments, the plurality of o-rings may be of varying sizes. In some embodiments, a smaller o-ring may provide a seal when the gate is perfectly aligned to either the “open” or “closed” position. In some embodiments, a second, larger o-ring prevents fluid leakage during the “swing” from “open” to “closed” position. See, for example, FIG. 19A.

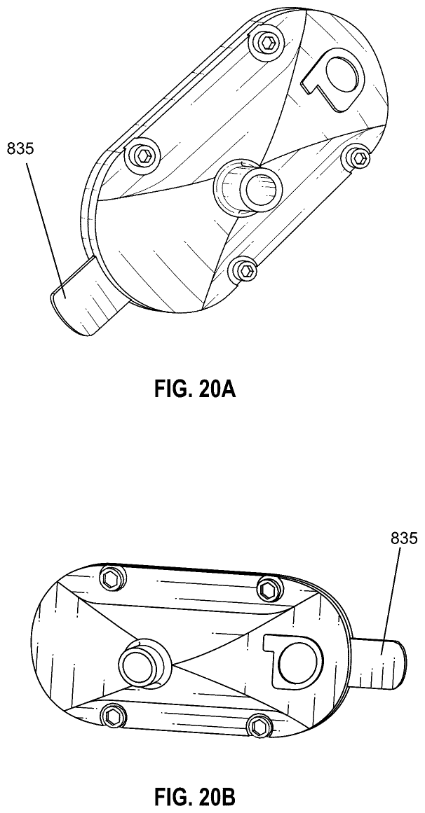

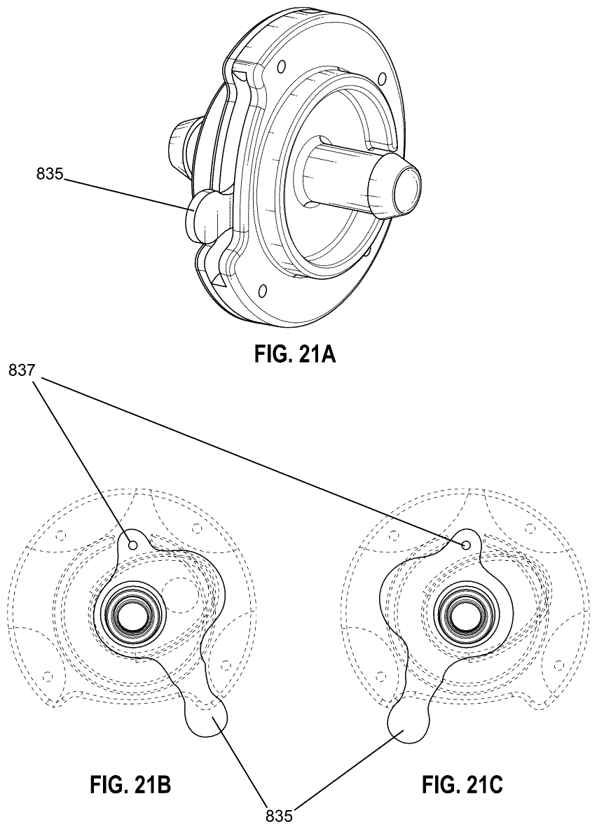

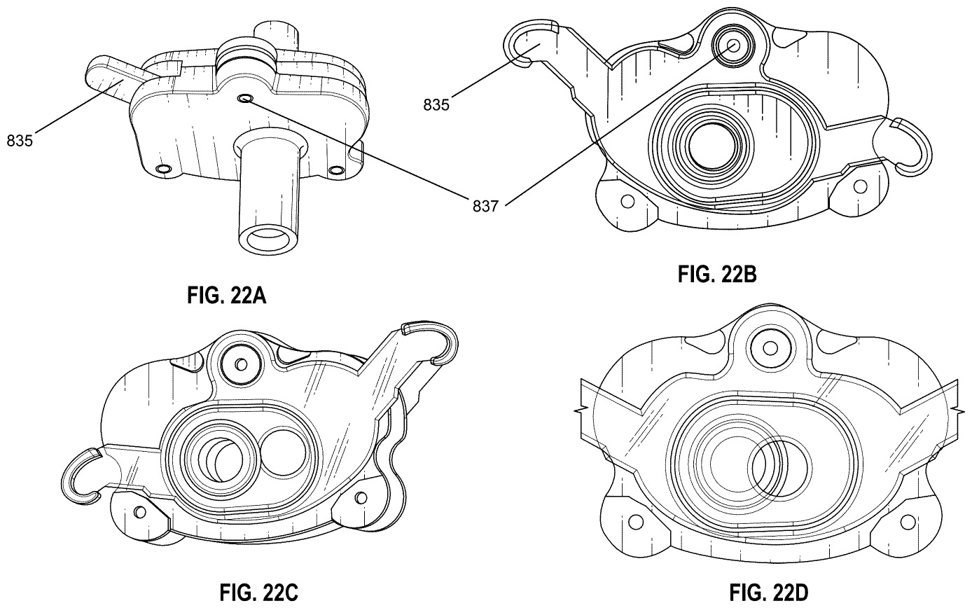

Gate valves may take on different embodiments. In some embodiments, the gate valve may be a “flat” style gate valve (see, for example, FIG. 19A-20B), which rely on linear motion of their gates relative to the ports within their valve bodies. In some embodiments, the gate valve may be a “rotational” style gate valve (see, for example, FIG. 21A-22D), which rely on rotational motion of their gates relative to the ports within their valve bodies. The gate rotates about a hinge point, which may allow for easier single handed operation.

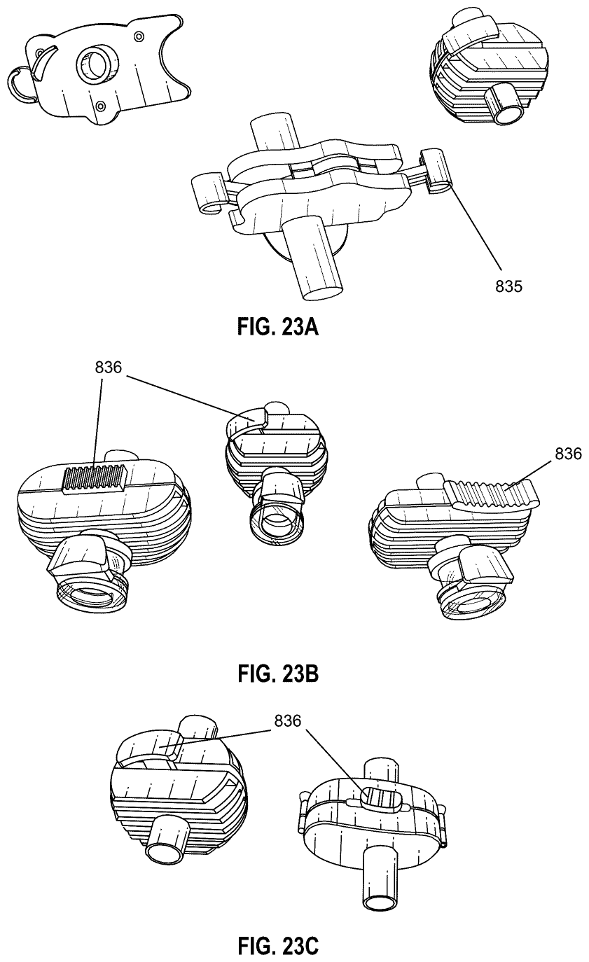

In some embodiments, the gate valve may be a “low profile” style gate valve (see, for example, FIGS. 23A-23C). Low profile style valves may have a smaller physical profile than other gate valves, and may optionally have a lever. In some embodiments, the lever may actuate the gate, moving it from the “lowered” (closed) position to the “raised” (open) position, and vice-versa.

In other embodiments, the alternative valve design may be a plunger valve. Plunger valves may optionally comprise o-rings to create a seal. O-rings may be used to compensate for manufacturing variations in molded components, thermal expansion of parts, and the like. The molded components of a plunger valve may all be made from the same material (e.g., hard plastic) because the design of the plunger valve negates the requirement of balancing between “soft” and “hard” materials that is present for traditional stopcock valves. Molding tolerances for plastic parts vary, but for the production of plunger valves in the present invention, can be considered to be in the range of about +/−0.003 inches. To allow for traditional mold tooling to create molded parts that may still achieve a secure seal, o-rings or other elastomeric components may compensate for dimensional variations in molded components due to natural variation in the molding process from unit-to-unit. The plunger of a plunger valve may be actuated in a variety of ways, most commonly by turning a knob operably connected to a plunger or to a valve stem, the valve stem disposed on an end of the plunger. The knob may be optionally equipped with knurling or other surface modifications to ease turning. The knob may be optionally bonded to the valve stem using adhesive. Alternatively, the knob may be secured to the valve stem via other mechanisms, for example, via screw(s).

Plunger valves of the present invention may be optionally equipped with a steel pin, typically pressed into the valve stem, and configured to ride in a pin groove cut into the valve body. In embodiments with a knob, when torque is applied to the knob, the pin will move in the pin groove. The pin groove is typically disposed on a position of the valve body, and shaped in such a way, that the groove guides the pin in such a way that the attached plunger is in turn lowered or elevated between the closed and open valve positions. In some embodiments, the pin groove may be configured such that a one-quarter (25%) turn of the knob or other valve plunger actuator results in complete actuation of the plunger valve. In some embodiments, the pin groove may be disposed at a roughly 45° angle. In embodiments with a knob(s), the knob may be optionally equipped with a skirt, the skirt configured to retain the pin, preventing the pin from migrating out of the valve stem and/or the plunger. The plunger may further comprise o-rings to enhance the seal between the plunger and the valve body. The o-rings may be lubricated to reduce the operational torque needed to open and close the valve.

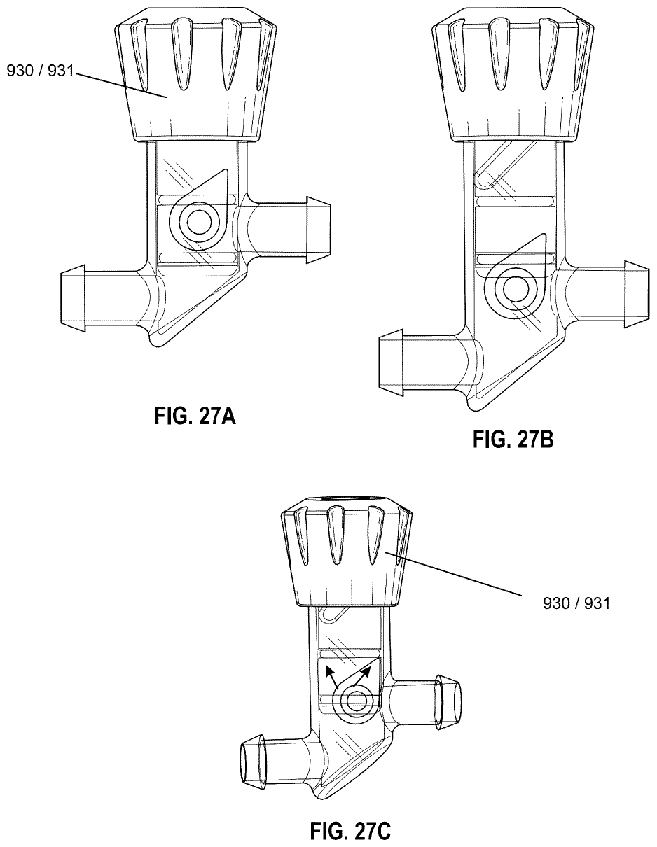

Plunger valves may contain o-ring(s). A plurality of o-rings may be used in plunger valves, using a similar theory of operation to that used in gate valves, as described elsewhere herein. In embodiments with multiple o-rings, one o-ring may serve to seal off one or more ports of the plunger valve while the plunger valve is in the closed position, while also fluidically coupling the one or more ports when in the open position. A second o-ring in the one or more o-rings may be configured to prevent leakage when moving the plunger between the open and closed positions. See, for example, FIG. 27A-27C.

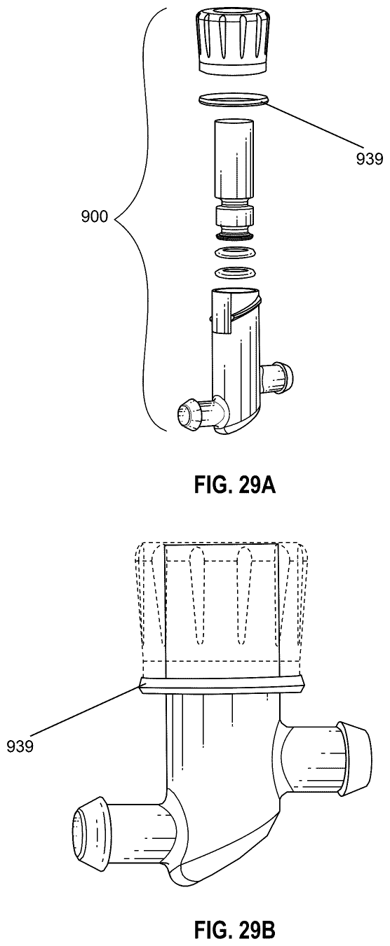

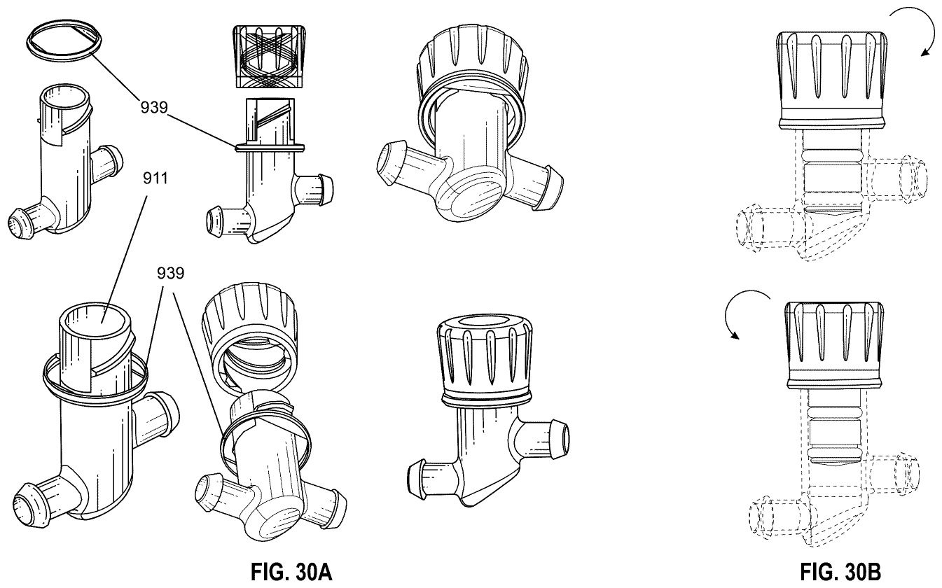

In some embodiments, the plunger valve may be configured to avoid lifting of the plunger and/or valve stem upon application of pressure to one or more of the plunger valve ports, including via incorporation of a stop limit device. In some embodiments, the plunger valve may be configured with a threaded knob or other stop limit device. In some embodiments, the knob may have internal threads and the valve body may have external threads, the knob's internal threads configured to releasably engage with the valve body's external threads. In some embodiments, the plunger valve may further comprise a knob bottom cap (939) configured to engage with the threaded knob (or otherwise engage with the knob) the bottom cap configured to act as a limit stop to prevent the valve stem and/or plunger from pushing the threaded knob off the valve body in the event that pressure from one or more of the plunger valve ports forces the valve stem and/or plunger to “raise” past the “open” position, wherein the application of enough such pressure (without the presence of the bottom cap or other stop limit device) might force the valve stem and/or plunger to separate from the valve body. In some embodiments, instead of reversibly mating via threading, the bottom cap is permanently fixed to the threaded knob (e.g., via chemical bonding or chemical adhesion, welding, or another appropriate, irreversible attachment methodology). See, for example, FIG. 29A-30B.

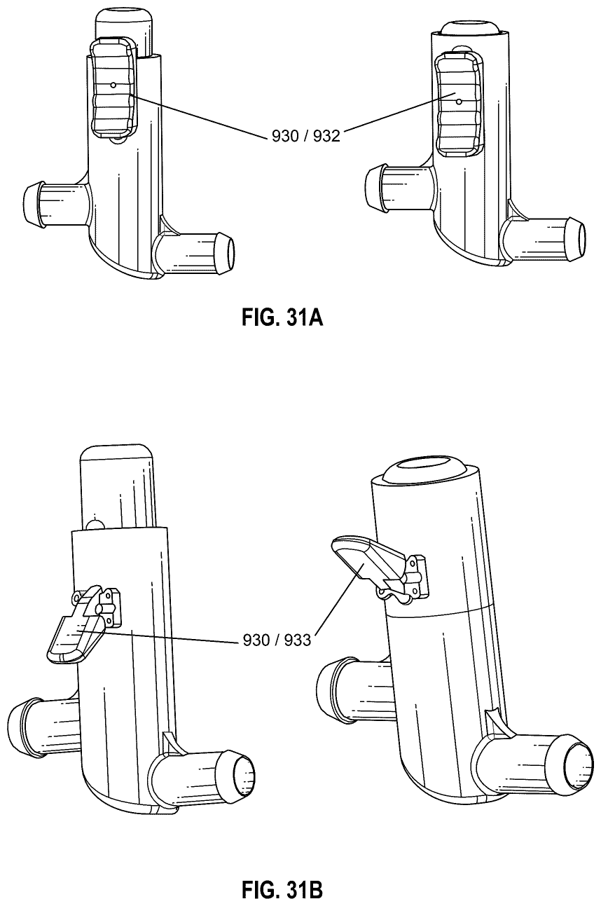

In other embodiments, the plunger valve may be actuated using alternative actuation mechanisms, including but not limited to slider switches or toggle switches, either configured to move the plunger from the “open” to “closed” position and vice-versa. See, for example, FIG. 31A-31B.

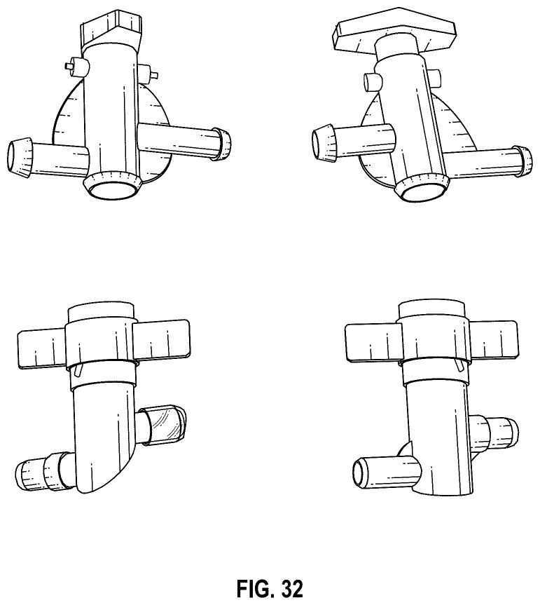

In other embodiments, the plunger valve may be equipped with an alternative actuation handle shape, instead of a traditional, round knob. See, for example, FIG. 32.

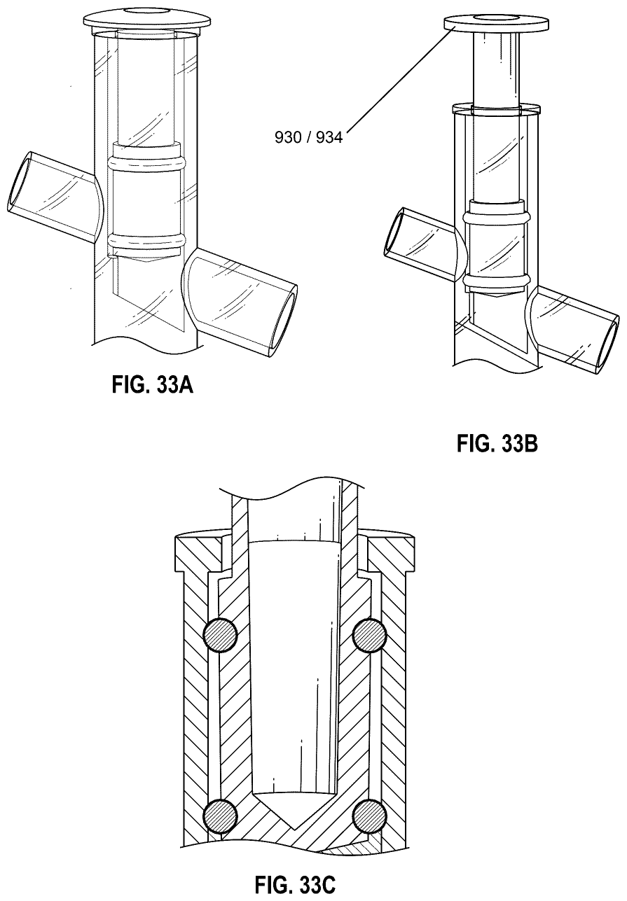

In some embodiments, plunger valves may be configured with an alternative “push-pull” valve actuation mechanism. In these embodiments, the plunger is actuated by pushing and/or pulling the plunger via either the plunger itself (with one end of the plunger protruding from the valve body, and thus manipulable by an operator) or a linearly-actuated handle disposed on one end of the plunger, the handle disposed coaxially with the plunger. The valve body may be optionally configured with a restraint edge or “lip” that prevents the push-pull plunger from separating from the valve body. See, for example, FIGS. 33A-33C.

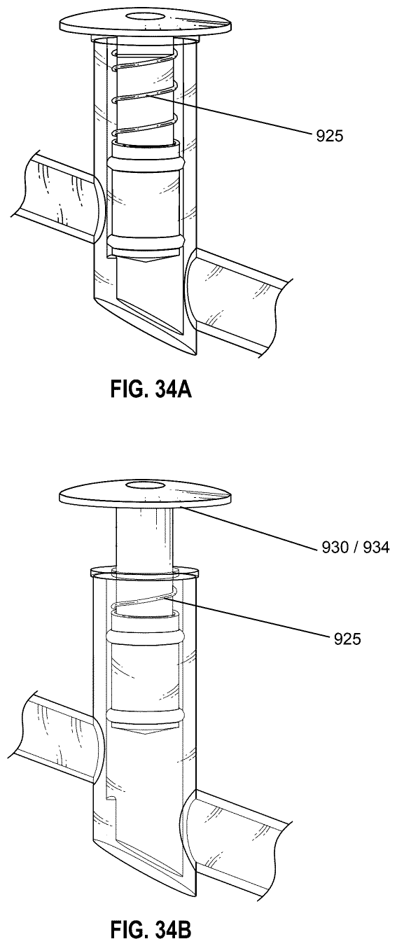

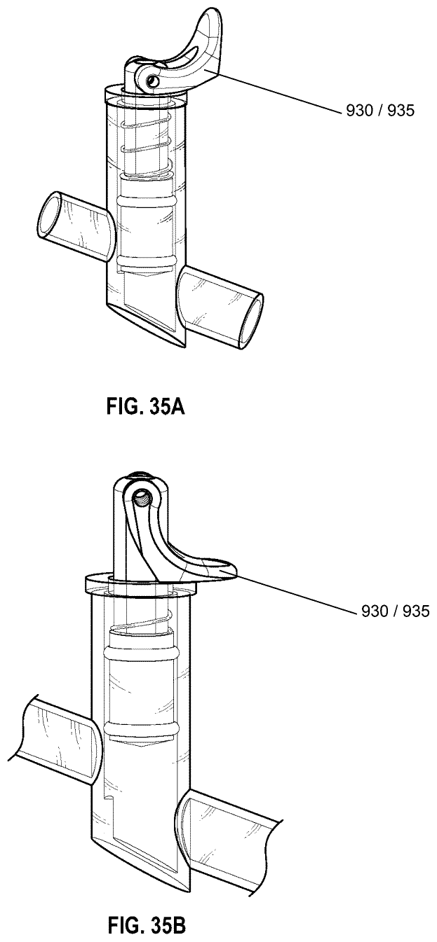

A “normally closed” valve, or, in other words, a valve that defaults (without user intervention) to a closed state provides benefits for use in thrombectomy procedures. Valve opening initiates aspiration. Aspiration timing is critical for aspirating the maximum amount of clot material. The clinician must steer the catheter to a target location where the clot resides. This positioning needs to be precisely timed with the initiation of aspiration to achieve optimal outcomes. A simple to use, “eyes-closed” valve actuation is therefore desired. Typically, closure of the valve is not nearly as important as opening of the valve, because closing of the valve only occurs after the timed steering and aspiration sequence has been initiated. For this reason, a normally closed fluid control device (e.g., a plunger valve, a stopcock, or the like) would be less confusing and easier to use for the clinician. See, for example, FIG. 34A-34B. The fluid control device may be kept in a normally closed position by use of a spring or other appropriate mechanical device. In the example of a normally closed plunger valve, a spring tension may be used to keep the plunger in a closed position unless aspiration is initiated by an operator actuating the plunger and overcoming the spring tension to move the plunger to an open position.

In some embodiments of a normally closed fluid control device, the fluid control device may be operated by a cam lever. The cam lever can be actuated by an operator to move the plunger (or other fluid flow restricting component) from its default “closed” state (as achieved by, for example, spring tension) to its open state. The valve body may optionally incorporate a stop limit to vertically align the plunger in its correct “closed” position within the valve body. See, for example, FIG. 35A-35B.

In still other embodiments, the stopcock may be substituted (or supplemented) with alternative closure mechanisms including but not limited to spring-loaded check valves, ball check valves, or rotatable hemostatic valves.

### EXAMPLE

The following is a non-limiting example of the present invention. It is to be understood that said example is not intended to limit the present invention in any way. Equivalents or substitutes are within the scope of the present invention.

Plunger valves of the present invention were tested to determine their performance. The following measurements were taken of one of said plunger valves. This plunger valve exceeded pressure performance tests of >60 psi, with no visual, tactile, or other indications of prolapse, indicating smooth operation. If an operation pressure target of 43 psi is chosen, results have indicated that a 10% o-ring compression provides satisfactory performance.

TABLE 1
“As built” measured geometry of plunger valve embodiment

| Plunger Valve Component | Measurement |
|---|---|
| Valve Stem OD | 0.503 inches |
| Valve Body ID | 0.515 inches |
| Unrestratined o-ring OD | 0.535 inches |
| O-ring groove OD | 0.340 inches |
| Radial projection | 0.016 inches |

TABLE 2
As Built Percent Compression of plunger valve embodiment

| | As-Built % Compression of plunger valve |
|---|---|
| Plunger Valve Component | embodiment |
| Radial gap | 0.006 inches |
| Radial o-ring compression | 0.010 inches |
| % compression | 10% |

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1) An vacuum-generating device (010) for intravascular aspiration of clot material, the device comprising:
a) a plunger (200) having a proximal plunger end (201) and a distal plunger end (202), the plunger (200) comprising one or more pawls (210) disposed at least partially around an outer circumference (203) of the plunger (200);
b) a chamber (100) having a proximal chamber end (101) and a distal chamber end (102), the plunger (200) slidably disposed within the chamber (100) and configured to form a fluid-tight seal with an inner wall of the chamber, the chamber (100) configured to generate a vacuum when the plunger (200) is drawn towards the proximal chamber end (101), the distal chamber end (102) having an aspiration port (120) disposed thereon;
c) a tension-generating member (300) having a proximal end (301) and a distal end (302), disposed within the chamber (100), the distal tension-generating member end (302) operatively connected to the proximal plunger end (201), the proximal tension-generating member end (301) operatively connected to a tension-generating member attachment point (310), the tension-generating member attachment point (310) disposed on or proximate to the proximal chamber end (101); the tension-generating member (300) configured to apply a force to the plunger (200) and draw the plunger (200) towards the proximal chamber end (101);
d) a ratcheting lever (400) having a proximal ratcheting lever end (401) and a distal ratcheting lever end (402), the distal ratcheting lever end (402) configured to engage the one or more pawls (210) of the plunger (200), the proximal ratcheting lever end (401) configured to move the distal ratcheting lever end (402) along the pawls (210) towards the proximal plunger end (201) when the proximal ratcheting lever end (401) is actuated; and
e) a ratchet locking member (500) configured to engage the one or more pawls (210), selectively hold the plunger (200) in a tensioned position, and release the plunger upon actuation of the ratchet locking member (500);
wherein the plunger (200), when drawn towards the distal chamber end (102), generates potential energy in the tension-generating member (300);
wherein a vacuum is generated and released via the aspiration port (120) thereby aspirating at least a portion of the clot material into the chamber (100) when the ratchet locking member (500) is actuated when the plunger (200) is in a tensioned position, thereby releasing the plunger (200) from its tensioned position and allowing it to travel towards the proximal chamber end (101), thereby generating a vacuum.

2) The vacuum-generating device of claim 1, further comprising an end cap (199) disposed on the proximal end (101) of the chamber (100).

3) The vacuum-generating device of claim 1, further comprising a fluid control device (050) disposed distally to the distal chamber end (102) and fluidly connected to the chamber (100) via the aspiration port (120).

4) A fluid decoupling damper (600) for use in a chamber (100) having a proximal chamber end (101) and a distal chamber end (102), the decoupling damper (600) having a proximal end (601) and a distal end (602), the decoupling damper (600) further comprising an energy-absorbing member (610) disposed on the decoupling damper (600) proximal end (601), the energy-absorbing member (610) configured to, upon aspiration of fluid into the chamber (100), at least partially absorb impact forces imparted to the decoupling damper (600) from the decoupling damper (600) making contact with the proximal end (101) of the chamber (100).

5) A slit valve assembly (700) for use in intravascular thrombectomy, comprising:
a) a seal housing (710) comprising a seal receptacle (715) disposed within the seal housing (710), a proximal housing end (711), a distal housing end (712), a proximal housing port (721) disposed on the proximal housing end (711), and a distal housing port (722) disposed on the distal housing end (712), the proximal housing port (721) fluidly connected to the seal receptacle (715), the seal receptacle (715) fluidly connected to the distal housing port (722), the distal housing port fluidly connected to a catheter (025);
b) an elastomeric seal (730) disposed in the seal receptacle (715), the elastomeric seal (730) having a slit (735) fluidly connecting a proximal elastomeric seal side (731) to a distal elastomeric seal side (732);
wherein, the elastomeric seal (730) is self-sealing when fluid is present on the distal elastomeric seal side (732) such that said fluid will not substantially leak from the distal elastomeric seal side (732) of the slit (735) onto the proximal elastomeric seal side (731);
wherein, when a hollow member (075) is inserted from the proximal elastomeric seal side (731) through the slit (735), fluid may flow from the catheter (025), through the slit (735), and into the hollow member (075).

6) The slit valve assembly (700) of claim 5, wherein the elastomeric seal (730) is operatively connected to one or more slit valve levers (750) and is configured to force the slit (735) into an open configuration when the one or more slit valve levers (750) are actuated by an operator.

7) The slit valve assembly (700) of claim 5, wherein the elastomeric seal (730) forms a tapered structure (774) that diverges proximally from the distal elastomeric seal side (732) and converges distally to the distal elastomeric seal side (732) to form an elongated tip (775) disposed distal to the distal elastomeric seal side (732), wherein one or more slit (735) is formed at the distal end of the elongated tip (775), and wherein the elongated tip (775) is symmetrical along at least one of its bilateral axes.

8) A gate valve assembly (800) for thrombectomy comprising:
a) a valve bonnet (810) having a valve bonnet opening (811);
b) a valve seat (820) having a valve seat opening (821); and
c) a valve gate (830) having a valve gate opening (831), the valve gate (830) configured to slidably fit between the valve bonnet (810) and valve seat (820);
wherein the valve gate (830) may be slidably positioned relative to the valve bonnet (810) and valve seat (820);
wherein, when the valve gate (830) is positioned such that valve gate opening (831) is substantially aligned with the valve bonnet opening (811) and valve seat opening (821), the valve bonnet opening (811), valve gate opening (831), and valve seat opening (821) are in fluid communication; and
wherein, when the valve gate (830) is positioned such that the valve gate opening (831), valve bonnet opening (811), and valve seat opening (821) are not substantially aligned, at least two of the valve gate (830), valve bonnet opening (811), and valve seat opening (821) are fluidically disconnected.

9) The gate valve assembly (800) of claim 8, wherein the valve gate (830) is operably connected to a valve gate lever (835), wherein the valve gate lever (835) is configured to slidably move the valve gate (830) relative to the valve bonnet (810) and valve seat (820) when the valve gate lever (835) is actuated.

10) The gate valve assembly (800) of claim 9, wherein the valve gate (830) is rotationally slidable around a valve gate hinge (837), the valve gate hinge (837) operably connecting the valve gate (830) to at least one of the valve bonnet (810) or valve seat (820).

11) The gate assembly (800) of claim 10, wherein the valve gate (830) is operably rotatable around the valve gate hinge (837) via actuation of a valve gate lever (835).

12) The gate assembly (800) of claim 11, wherein the valve gate lever (835) comprises a low-profile switch (836).

13) A plunger valve assembly (900) for use in intravascular thrombectomy comprising:
a) a valve body (910) comprising an valve cavity (911) disposed within the valve body (910), a plunger seat (912) disposed at the proximal terminus of the valve cavity (911), and at least two plunger valve ports (915) fluidly connected to the valve cavity (911);
b) a valve plunger (920) configured to slidably fit within the valve cavity (911);
c) a valve plunger actuator (930) disposed at the distal terminus of the valve plunger (921) and configured to, upon actuation, move the valve plunger (920) between a first valve plunger position and a second valve plunger position;
wherein, the valve plunger (920) fluidically disconnects the at least two plunger valve ports (915) from each other when in the first plunger position;
wherein the at least two plunger valve ports (915) are fluidically connected when the plunger valve is in the second plunger position.

14) The plunger valve assembly (900) of claim 13, wherein the valve plunger actuator (930) comprises a rotatable knob (931).

15) The plunger valve assembly (900) of claim 13, wherein the valve plunger actuator (930) comprises a low-profile slider switch (932).

16) The plunger valve assembly (900) of claim 13, wherein the valve plunger actuator (930) comprises a toggle switch (933).

17) The plunger valve assembly (900) of claim 13, wherein the valve plunger actuator (930) comprises a linearly-actuated handle (934).

18) The plunger valve assembly (900) of claim 13, wherein the valve plunger actuator (930) comprises a cam lever (935).

19) The plunger valve assembly (900) of claim 13, wherein the plunger body (910) further comprises a pin groove (951), and wherein the valve plunger (920) further comprises a pin (950), wherein the pin (950) is configured to slidably fit within the pin groove (951).

20) The plunger valve assembly (900) of claim 13, wherein the valve plunger (920) comprises a normally-closed plunger, wherein the valve plunger (920) is held in its normally closed position via spring tension provided by a spring (925).
